(19) United States
(12) Patent Application Publication
(10) Pub. No.: US 2014/0051709 A1
(43) Pub. Date: Feb. 20, 2014

(54) Compositions and Methods for Treating Myotonic Dystrophy Type 1

(71) Applicants: Sita REDDY, Pasadena, CA (US); Lucio Comai, Pasadena, CA (US); Nouri Neamati, Fullerton, CA (US)

(72) Inventors: Sita REDDY, Pasadena, CA (US); Lucio Comai, Pasadena, CA (US); Nouri Neamati, Fullerton, CA (US)

(73) Assignee: UNIVERSITY OF SOUTHERN CALIFORNIA, Los Angeles, CA (US)

(21) Appl. No.: 13/587,858

(22) Filed: Aug. 16, 2012

(51) Int. Cl. A61K 31/366; A61K 31/4196; C07D 413/04; A61K 31/4245; C07D 401/04; A61P 21/00; C07D 311/16; C07D 405/06; A61K 31/496; C07D 417/04; A61K 31/427; C07D 405/04; A61K 31/437

(52) U.S. Cl. 514/254.11; 548/266.4; 514/383; 548/143; 514/364; 546/121; 514/300; 549/287; 514/456; 544/376; 548/204; 514/365

(57) ABSTRACT

Disclosed is a compound for treatment of Myotonic Dystrophy type 1 having the formula:

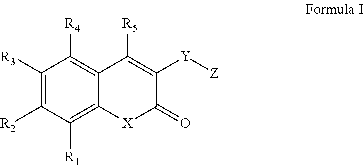

Wherein X is selected from the group consisting of O, N, C, or S, Y is a homo- or heteroatomic 5-membered ring comprising one or more atoms selected from the group consisting of N, O, S, and C, Z is an optionally substituted aryl group or optionally substituted heteroaryl, including but not limited to halogenated benzenes, pyridines, substituted benzene, substituted pyridine, R₂=hydroxy, acyl, alkoxyl, esters, ethers, cyclic ethers, and lactones, R₃=H, alkyl, an optionally substituted alkyl, aliphatic ether, ester, cyclic unsaturated and aromatic ring groups, and R₁, R₄ and R₅ are independently selected from the group consisting of hydrogen, halogen, alkyl, and alkoxyl or a pharmaceutically or cosmetically acceptable salt, solvate, or hydrate thereof.

### STATEMENT REGARDING FEDERALLY SPONSORED RESEARCH OR DEVELOPMENT

This invention was made with government support under Contract Nos. 5R01NS060839 and 5R01NS050861 awarded by the National Institute for Health. The government has certain rights in the invention.

### FIELD OF THE INVENTION

The present invention relates to compositions and methods of treating Myotonic Dystrophy type 1.

### BACKGROUND OF THE INVENTION

Myotonic Dystrophy type 1 (DM1) is the most common form of muscular dystrophy diagnosed in adults, with a prevalence ranging from 1 per 100,000 in Japan to 3-15 per 100,000 in Europe. The worldwide incidence is estimated to be approximately 1 per 8,000. DM1 patients often present with myotonia, disabling distal weakness and severe cognitive problems.

DM1 is an autosomal dominant disorder resulting from the expansion of a noncoding (CTG)n repeat expansion located in the 3′ untranslated region of DMPK (dystrophia myotonica protein kinase) gene (1). Expansion of the CTG tract is a key defect in DM1, as context independent expression of CTGexp in the HSA^{LR} mice, which encode an expanded CTG tract in the 3′UTR of the human skeletal actin gene, develop DM1 skeletal muscle pathology (9). In other experiments, transgenic mouse models, which are designed to express CTGexp in an inducible manner, show DM1 pathology only when transcription is induced (10). More recently, AON mediated degradation of the CUGexp RNA in DM1 mouse models has been shown to reverse DM1 pathology (7). Thus these experiments, taken together, demonstrate that CUGexp RNA is a central target for DM1 therapy.

Expression of CUGexp RNA causes aberrant sequestration of the RNA splice regulator, muscleblind 1 (MBNL1) in nuclear CUG-RNA-protein aggregates or foci in DM1 cells (2,3,4). Therefore, a major consequence of the expression of toxic CUGexp RNA is the abnormal splicing of a set of physiologically important RNAs, such as the chloride channel and the IR receptor that have been implicated in the development of myotonia and insulin resistance in DM1 patients (5,6). Importantly, functional inactivation of Mbnl1 in mice recapitulates key features of DM1 pathology (11) and transcriptome analysis demonstrates that >80% of the splice defects observed in the HSA^{LR} mice are identical to those observed in Mbnl1 knockout mice (12). In addition to splice defects, expression of CUGexp RNA results in the increased steady-state levels of CUG-BP1, activation of PKC alpha (13), mis-localization of the transcription factor, SHARP, from the nucleus to the cytoplasm (14) and ectopic expression of NKX2.5 in DM1 myoblasts (15). Therefore in addition to splice defects these molecular defects serve as biomarkers for DM1.

Warf and colleagues have demonstrated that pentamidine, which was identified using a candidate molecule approach from a pool of 26 compounds, facilitates a partial rescue of DM1 pathology (16). In other studies use of AON that hybridize to CUGexp RNA have been shown to displace MBNL1, disperse the CUG foci and significantly reverse the splice defects in the HSA^{LR} DM1 mouse model (17). However, therapeutic use of AON especially in the CNS faces significant technical hurdles.

There is currently no cure for or treatment specific to myotonic dystrophy, and the clinical focus is on managing the complications of the disease, particularly those relating to the cardiopulmonary system as these account for 70% of deaths due to DM1. As such, there is a continuing need for compositions and methods which can treat myotonic dystrophy type 1.

### SUMMARY OF THE INVENTION

One object of the present invention is to provide compounds useful for the treatment of Myotonic Dystrophy 1.

Another object of the present invention is to provide pharmaceutical compositions useful the treatment of Myotonic Dystrophy 1.

Another object of the present invention is to provide methods of treating a patient having Myotonic Distrophy 1.

One embodiment of the present invention is directed to a compound having the formula:

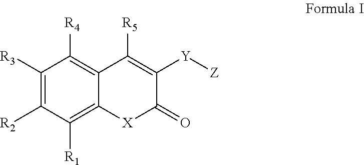

wherein

X is selected from the group consisting of O, N, C, or S,

Y is a homo- or heteroatomic 5-membered ring comprising one or more atoms selected from the group consisting of N, O, S, and C, including but not limited to thiazoles, oxadiazoles, triazoles,

Z is an optionally substituted aryl group or optionally substituted heteroaryl, including but not limited to halogenated benzenes, pyridines, substituted benzene, substituted pyridine,

R₂=hydroxy, acyl, alkoxyl, esters, ethers, cyclic ethers, and lactones.

R₃=H, alkyl, an optionally substituted alkyl, aliphatic ether, ester, cyclic unsaturated and aromatic ring groups, and

R₁, R₄ and R₅ are independently selected from the group consisting of hydrogen, halogen, alkyl, and alkoxyl,

or a pharmaceutically or cosmetically acceptable salt, solvate, or hydrate thereof.

Another embodiment of the present invention is directed to a compound having a formula:

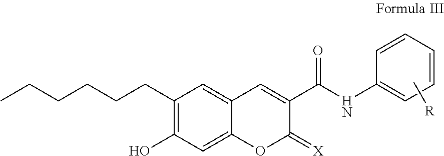

where R indicates a variable substitution of the 6-membered ring and includes mono-, di-, tri-, tetra- and penta-substituted embodiments wherein R is independently selected from the group consisting of halogen, alkyl, alkoxyl, substituted benzene; and

X=O or N, or

a pharmaceutically or cosmetically acceptable salt, solvate, or hydrate thereof.

Another embodiment of the present invention is directed to a pharmaceutical composition comprising the compound according to the present invention dispersed or dissolved in a pharmaceutically acceptable carrier.

Another embodiment of the present invention is directed to a method of treating myotonic dystrophy type 1 comprising administering to a subject in need thereof an effective amount of the pharmaceutical composition comprising a carrier and the compounds according to the present invention.

Another embodiment of the present invention is directed to a method of preventing the progression of DM1 symptoms comprising administering to a subject in need thereof a therapeutically effective amounts of a pharmaceutical composition comprising a carrier and the compound according to present invention.

Another embodiment of the present invention is directed to a method of preventing the onset of DM1 symptoms comprising administering to a subject in need thereof a therapeutically effective amounts of a pharmaceutical composition comprising a carrier and the compounds of the present invention.

### BRIEF DESCRIPTION OF THE FIGURES

The file of this patent contains at least one drawing executed in color. Copies of this patent with color drawings will be provided by the Office upon request and payment of the necessary fee.

FIG. 1 shows A. Representative images of RNA foci in control and RCN-001-treated DM1 myoblasts. Panels 1 and 4 show DAPI staining of DM1 myoblast nuclei; Panels 2 and 5 show FISH analysis of CUG-containing RNA foci in control DM1 myoblasts treated with vehicle (DMSO) (panels 1-3) or 10 μM RCN-001 for 4 days (panels 4-6) using (CAG)₁₀Cy3 labeled probe. Panels 3 and 6 show an overlay of images. Bars=20 μM B. DM1 myoblasts were treated with 10 μM RCN-001 for 4 days, and CUG RNA foci were visualized and counted. DM1 myoblasts showing 1, 2 and >3 CUG foci are shown as percentage of total cells.

FIG. 2 shows that treatment with RCN-001 rescues abnormal RNA splicing in DM1 myoblasts. A. Total RNA was isolated from normal (SKMC) myoblasts, untreated DM1 myoblasts and DM1 myoblasts treated with 50 μM RCN-001 or 50 μM Pentamidine for 17 hours and subjected to RT-PCR analysis using the indicated primers sets for MBNL1 and MBNL2 RNAs. GAPDH RNA was amplified in parallel as an internal control. The results of one of three experiments are shown. B. The relative levels of exon inclusion was measured by densitometry and tabulated as mean % exon inclusion +/− standard deviation.

FIG. 3 shows that treatment with RCN-001 reduces CUG foci load in HSA^{LR} mice. Red: CUG foci, detected with a (CAG)₁₀Cy3 probe Blue: DAPI stain of nuclei.

FIG. 4 shows that treatment with RCN-001 rescues abnormal RNA splicing in HSA^{LR} mice. A. Total RNA was isolated from age and gender matched wild-type mice, HSA^{LR} mice and HSA^{LR} mice treated with either saline or 40 mgs/kg of RCN-001 injected i.p. once a day for 7 days. Subsequent to treatment RNA was isolated from skeletal muscles and subjected to RT-PCR analysis using the indicated primers sets for Clcn1, Lbd3 and Serca-1 RNAs. GAPDH RNA was amplified in parallel as an internal control. The results of two experiments are shown. B. Bar graph representation of rescue achieved is shown. C. The relative levels of exon inclusion was measured by densitometry and tabulated as mean % exon inclusion +/− standard deviation.

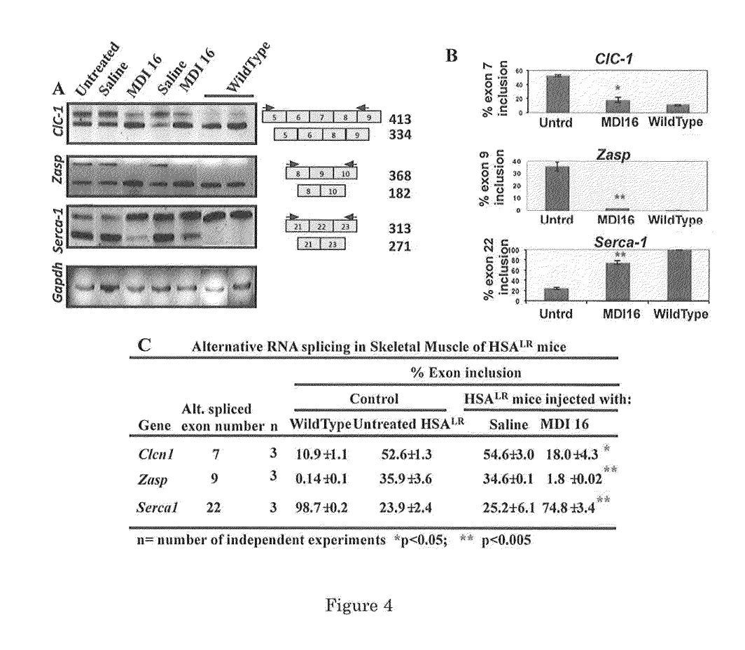

FIG. 5 shows A: HSA^{LR} mice were treated with either vehicle (saline) or 40 mgs/kg of RCN-001 injected IP once a day for 14 days and examined for myotonic runs≧1 second in the gastrocnemius muscle. For all animals the recording electrode was inserted into the test muscle 20 times. The number of myotonic runs≧1 second in the gastrocnemius muscle of individual HSA^{LR} mice treated with either the saline control or RCN-001 are shown. B: Sections of the gastrocnemius muscle stained with anti-Clcn1 antibodies, demonstrates that the decrease in Clcn1 levels observed in the HSA^{LR} mice (5) are rescued by RCN-001 treatment.

FIG. 6 shows A: RCN-001 treatment does not alter splicing of RNAs that are normally spliced in DM1 myoblasts B. RCN-001 treatment rescues aberrant splicing of MBNL1, MBNL2, and IR in DM1 myoblasts, but does not alter the splicing of these RNAs in normal myoblasts. C: RCN-001 treatment does not significantly alter steady-state levels of endogenous CUG repeat containing transcripts in normal and DM1 myoblasts. Semi-quantitative RT-PCR was done as previously described (7, 17, 23). Abbreviations: U: Untreated; V: vehicle treated; 16: treated with RCN-001.

### DETAILED DESCRIPTION OF THE INVENTION

### Definitions

Unless otherwise indicated herein, all terms used herein have the meanings that the terms would have to those skilled in the art of the present invention. Practitioners are particularly directed to current textbooks for definitions and terms of the art. It is to be understood, however, that this invention is not limited to the particular methodology, protocols, and reagents described, as these may vary.

The term “alkyl” herein used means C₁-C₁₀ straight or branched chain alkyl, for example, methyl, ethyl, n-propyl, i-propyl, n-butyl, i-butyl, sec-butyl, tert-butyl, n-pentyl, i-pentyl, neo-pentyl, tert-pentyl, and the like.

The term “alkoxy” or “alkoxyl” herein used means alkoxy of which alkyl part is the above mentioned alkyl. Examples of the alkoxy are methoxy, ethoxy, propoxy, butoxy, pentyloxy, and the like.

The term “acyl” refers to an alkylcarbonyl, cycloalkylcarbonyl, arylcarbonyl, heterocyclylcarbonyl, or heteroarylcarbonyl substituent, any of which can be further substituted, e.g., by one or more substituents.

The term “halogen” herein used means fluoro, chloro, bromo, and iodo.

The term “ester” includes compounds and moieties which contain a carbon or a heteroatom bound to an oxygen atom which is bonded to the carbon of a carbonyl group.

The term “ester” includes alkoxycarboxy groups such as methoxycarbonyl, ethoxycarbonyl, propoxycarbonyl, butoxycarbonyl, pentoxycarbonyl, etc.

The term “ether” includes compounds or moieties which contain an oxygen bonded to two different carbon atoms or heteroatoms.

The term “aliphatic” or “aliphatic group”, as used herein, means a straight-chain (i.e., unbranched) or branched, substituted or unsubstituted hydrocarbon chain that is completely saturated or that contains one or more units of unsaturation.

The term “cyclic unsaturated group” means 3 to 8 membered cyclic or heterocyclic ring which, when heterocyclic, contains one or more hetero atoms selected from the group consisting of nitrogen, oxygen and sulfur atoms in the ring, and may bind at any possible position and which has at least one site of unsaturation.

The term “aryl” herein used means monocyclic or condensed ring aromatic hydrocarbons. Examples of the aryl are phenyl, naphthyl, and the like. The aryl may be optionally substituted.

The term “heteroaryl” herein used means a 5 to 6 membered aromatic heterocyclic group which contains one or more hetero atoms selected from the group consisting of nitrogen, oxygen and sulfur atoms in the ring and may be fused with a carbocyclic ring or other heterocyclic ring at any possible position. The heteroaryl may be optionally substituted.

Substituents for “optionally substituted alkyl” are hydroxy, alkoxy (e.g., methoxy and ethoxy), mercapto, alkylthio (e.g., methylthio), cycloalkyl (e.g., cyclopropyl, cyclobutyl, cyclopentyl, and cyclohexyl), halogen (e.g., fluoro, chloro, bromo, and iodo), carboxy, alkoxycarbonyl (e.g., methoxycarbonyl and ethoxycarbonyl), nitro, cyano, haloalkyl (e.g., trifluoromethyl), substituted or unsubstituted amino (e.g., methylamino, dimethylamino, and carbamoylamino), guanidino, phenyl, benzyloxy, and the like. These substituents are able to bind them at one or more of any possible positions.

Substituents for the “optionally substituted aryl” and “optionally substituted heteroaryl” are, for example, hydroxy, alkoxy (e.g., methoxy and ethoxy), mercapto, alkylthio (e.g., methylthio), cycloalkyl (e.g., cyclopropyl, cyclobutyl, cyclopentyl), halogen (e.g., fluoro, chloro, bromo, and iodo), carboxy, alkoxycarbonyl (e.g., methoxycarbonyl and ethoxycarbonyl), nitro, cyano, haloalkyl (e.g., trifluoromethyl), aryloxy (e.g., phenyloxy) substituted or unsubstituted amino (e.g., methylamino, dimethylamino, diethylamino, and benzylidenamino), guanidine, alkyl (e.g., methyl, ethyl, n-propyl, i-propyl, n-butyl, i-butyl, sec-butyl, tert-butyl, n-pentyl, i-pentyl, neo-pentyl, and tert-pentyl), alkenyl (e.g., vinyl and propenyl), alkynyl (e.g., ethynyl and phenylethynyl), alkanoyl (e.g., formyl, acetyl, and propionyl), acyloxy (e.g., acetyloxy), acylamino, alkylsulfonyl (e.g., methylsulfonyl), phenyl, benzyl, an azo group (e.g., phenylazo), optionally substituted heteroaryl (e.g., 3-pyridyl), optionally substituted ureido (e.g., ureido and phenylureido), and the like. These substituents are able to bind to it at one or more of any possible position.

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

The compounds herein may be referred to either by a chemical formula, a chemical name or by an assigned reference number of the form RCN-XXX. If there is any incidental conflict between formula, chemical name or reference name, the formula shall control.

The compounds according to the present invention rescue DM1 pathology. One embodiment of the present invention is directed to a compound having the formula:

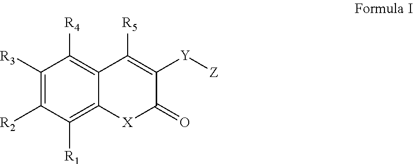

wherein

X is selected from the group consisting of O, N, C, or S,

Y is a homo- or heteroatomic 5-membered ring comprising one or more atoms selected from the group consisting of N, O, S, and C,

Z is an optionally substituted aryl group or optionally substituted heteroaryl, including but not limited to halogenated benzenes, pyridines, substituted benzene, substituted pyridine,

R₂=hydroxy, acyl, alkoxyl, esters, ethers, cyclic ethers, and lactones,

R₃=H, alkyl, an optionally substituted alkyl, aliphatic ether, ester, cyclic unsaturated and aromatic ring groups, and

R₁, R₄ and R₅ are independently selected from the group consisting of hydrogen, halogen, alkyl, and alkoxyl,

or a pharmaceutically or cosmetically acceptable salt, solvate, or hydrate thereof.

In a preferred embodiment, R₁ and R₄ are hydrogen.

In another preferred embodiment, Y is selected from the group consisting of thiazoles, oxadiazoles and triazoles.

In another preferred embodiment, R₁ and R₄ are hydrogen, and Y is selected from the group consisting of thiazoles, oxadiazoles and triazoles.

It should be noted that when X is selected to be N or C, the substituted group may be NR′ or CR′₂ where R′ is selected from the group consisting of hydrogen, alkyl and optionally substituted alkyl.

In a preferred embodiment, the compounds according the present invention have the formula:

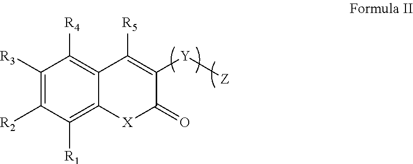

### Wherein X is O, N, C, or S,

R₁ is hydrogen,
R₄ is hydrogen,
Y comprises a five membered heterocyclic ring selected from the group consisting of

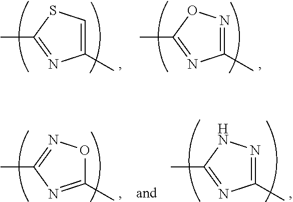

Z is a pyridyl of phenyl group, preferably selected from the group consisting of

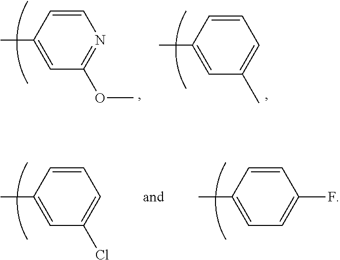

R₂=hydroxy, acyl, alkoxyl, esters, ethers, cyclic ethers, and lactones,

R₃=H, alkyl, an optionally substituted alkyl, aliphatic ether, ester, cyclic unsaturated and aromatic ring groups, and

R₅ are independently selected from the group consisting of hydrogen, halogen, alkyl, and alkoxyl, or a pharmaceutically or cosmetically acceptable salt, solvate, or hydrate thereof.

In a preferred embodiment, the compound according to the present invention is selected from of the following, or a pharmaceutically or cosmetically acceptable salt, solvate, or hydrate thereof:

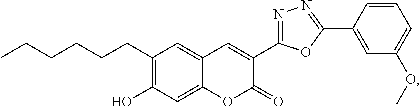 [SMILES: CCCCCCc1cc2cc(-c3nnc(-c4cccc(OC)c4)o3)c(=O)oc2cc1O]

6-hexyl-7-hydroxy-3-(5-(3-methoxyphenyl)-1,3,4-oxadiazol-2-yl)-4-methyl-2H-chromen-2-one, referred to herein as “RCN-001,”

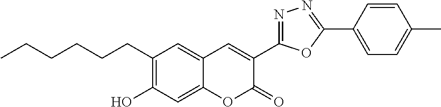 [SMILES: CCCCCCc1cc2cc(-c3nnc(-c4ccc(C)cc4)o3)c(=O)oc2cc1O]

6-hexyl-7-hydroxy-3-(5-(p-tolyl)-1,3,4-oxadiazol-2-yl)-2H-chromen-2-one, referred to herein as “RCN-002,”

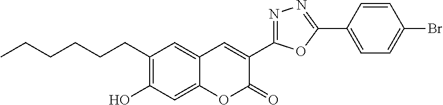 [SMILES: CCCCCCc1cc2cc(-c3nnc(-c4ccc(Br)cc4)o3)c(=O)oc2cc1O]

3-(5-(4-bromophenyl)-1,3,4-oxadiazol-2-yl)-6-hexyl-7-hydroxy-2H-chromen-2-one, referred to herein as “RCN-003”

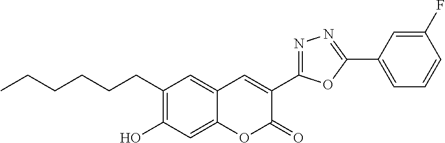 [SMILES: CCCCCCc1cc2cc(-c3nnc(-c4cccc(F)c4)o3)c(=O)oc2cc1O]

3-(5-(3-fluorophenyl)-1,3,4-oxadiazol-2-yl)-6-hexyl-7-hydroxy-2H-chromen-2-one, referred to herein as “RCN-004,”

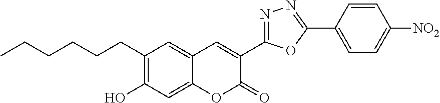 [SMILES: CCCCCCc1cc2cc(-c3nnc(-c4ccc([N+](=O)[O-])cc4)o3)c(=O)oc2cc1O]

6-hexyl-7-hydroxy-3-(5-(4-nitrophenyl)-1,3,4-oxadiazol-2-yl)-2H-chromen-2-one, referred to herein a “RCN-005,”

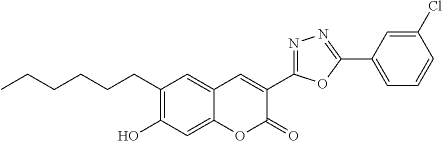 [SMILES: CCCCCCc1cc2cc(-c3nnc(-c4cccc(Cl)c4)o3)c(=O)oc2cc1O]

3-(5-(3-chlorophenyl)-1,3,4-oxadiazol-2-O-6-hexyl-7-hydroxy-2H-chromen-2-one, referred to herein as “RCN-006,”

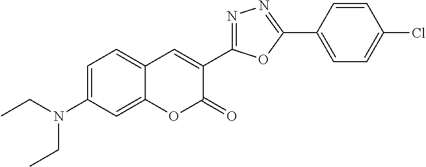 [SMILES: CCN(CC)c1ccc2cc(-c3nnc(-c4ccc(Cl)cc4)o3)c(=O)oc2c1]

3-(5-(4-chlorophenyl)-1,3,4-oxadiazol-2-yl)-7-(diethylamino)-2H-chromen-2-one, referred to herein as “RCN-007,”

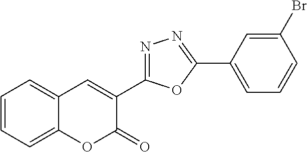 [SMILES: O=c1oc2ccccc2cc1-c1nnc(-c2cccc(Br)c2)o1]

3-(5-(3-bromophenyl)-1,3,4-oxadiazol-2-yl)-2H-chromen-2-one, referred to herein as “RCN-008,”

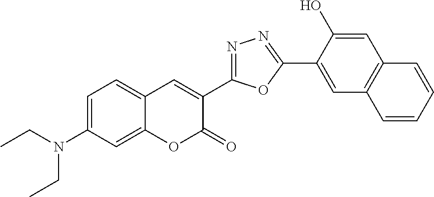 [SMILES: CCN(CC)c1ccc2cc(-c3nnc(-c4cc5ccccc5cc4O)o3)c(=O)oc2c1]

7-(diethylamino)-3-(5-(3-hydroxynaphthalen-2-yl)-1,3,4-oxadiazol-2-yl)-2H-chromen-2-one, referred to herein as “RCN-009,”

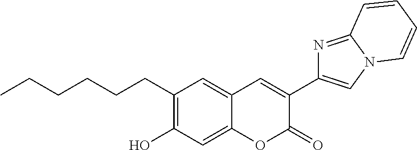 [SMILES: CCCCCCc1cc2cc(-c3cn4ccccc4n3)c(=O)oc2cc1O]

6-hexyl-7-hydroxy-3-(imidazo[1,2-a]pyridine-2-yl)-2H-chromen-2-one, referred to herein as “RCN-011,”

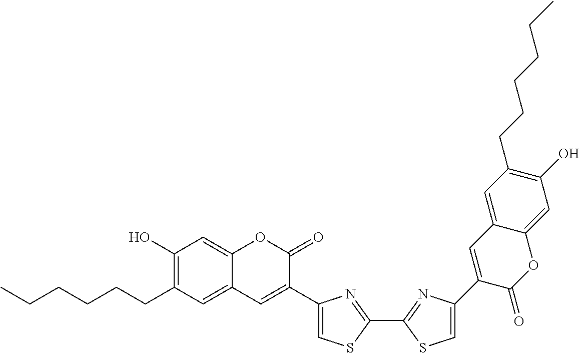 [SMILES: CCCCCCc1cc2cc(-c3csc(-c4nc(-c5cc6cc(CCCCCC)c(O)cc6oc5=O)cs4)n3)c(=O)oc2cc1O]

3,3′-([2,2′-bithiazole]-4,4′-diyl)bis(6-hexyl-7-hydroxy-2H-chromen-2-one, referred to herein as “RCN-012,” and

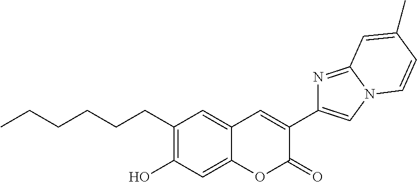 [SMILES: CCCCCCc1cc2cc(-c3cn4ccc(C)cc4n3)c(=O)oc2cc1O]

6-hexyl-7-hydroxy-3-(7-methylimidazo[1,2-a]pyridine-2-yl)-2H-chromen-2-one, Referred to herein as “RCN-013,”
   or a pharmaceutically or cosmetically acceptable salt, solvate, or hydrate thereof.

Another embodiment of the present invention is directed to a compound having a formula:

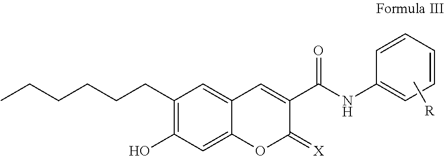

where R indicates a variable substitution of the 6-membered ring and includes mono-, di-, tri-, tetra- and penta-substituted embodiments wherein R is independently selected from the group consisting of halogen, alkyl, alkoxyl, substituted benzene; and

X=O or N, or

a pharmaceutically or cosmetically acceptable salt, solvate, or hydrate thereof.

It should be noted that when X is selected to be N, the substituted group may be NR′ where R′ is selected from the group consisting of hydrogen, alkyl and optionally substituted alkyl.

In a preferred embodiment, a compound according to the present invention is selected from the group consisting of:

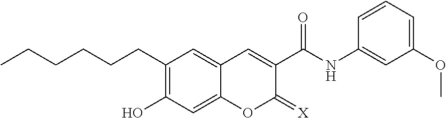

6-hexyl-7-hydroxy-N-(3-methoxyphenyl)-2-oxo-2H-chromene-3-carboxamide, referred to herein as “RCN-014,”

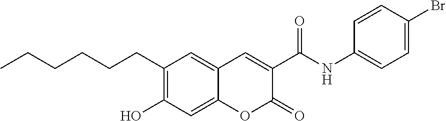 [SMILES: CCCCCCc1cc2cc(C(=O)Nc3ccc(Br)cc3)c(=O)oc2cc1O]

N-(4-bromophenyl)-6-hexyl-7-hydroxy-2-oxo-2H-chromene-3-carboxamide, referred to herein as “RCN-015,”

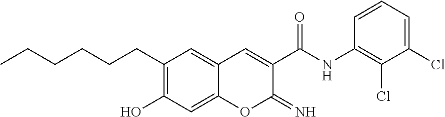 [SMILES: CCCCCCc1cc2cc(C(=O)Nc3cccc(Cl)c3Cl)c(=N)oc2cc1O]

N-(2,3-dichlorophenyl)-6-hexyl-7-hydroxy-2-imino-2H-chromene-3-carboxamide, hereinafter referred to as “RCN-016,”

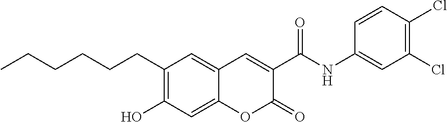 [SMILES: CCCCCCc1cc2cc(C(=O)Nc3ccc(Cl)c(Cl)c3)c(=O)oc2cc1O]

N-(3,4-dichlorophenyl)-6-hexyl-7-hydroxy-2-oxo-2H-chromene-3-carboxamide, referred to herein as “RCN-017,” and

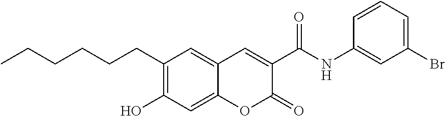 [SMILES: CCCCCCc1cc2cc(C(=O)Nc3cccc(Br)c3)c(=O)oc2cc1O]

N-(3-bromophenyl)-6-hexyl-7-hydroxy-2-oxo-2H-chromene-3-carboxamide, referred to herein as “RCN-018”,

or a pharmaceutically or cosmetically acceptable salt, solvate, or hydrate thereof. The 2-oxo-2H-chromene-3-carboxamide compositions of Formula II may be made according to methods known to those of ordinary skill.

In another embodiment of the present invention, a compound according to the present invention is selected from the group consisting of:

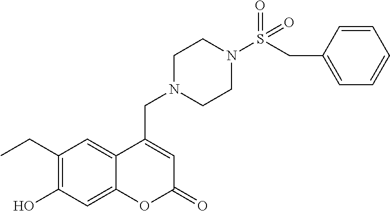 [SMILES: CCc1cc2c(CN3CCN(S(=O)(=O)Cc4ccccc4)CC3)cc(=O)oc2cc1O]

4-((4-(benzylsulfonyl)piperazin-1-yl)methyl)-6-ethyl-7-hydroxy-2H-chromen-2-one, referred to herein as “RCN-020,”

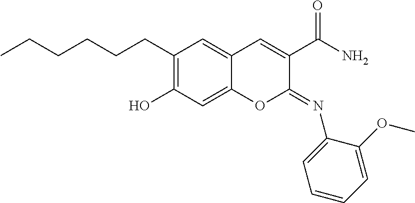 [SMILES: CCCCCCc1cc2cc(C(N)=O)/c(=N/c3ccccc3OC)oc2cc1O]

(Z)-6-hexyl-7-hydroxy-2-((2-methoxyphenyl)imino)-2H-chromene-3-carboxamide, referred to herein as “RCN-019”, and

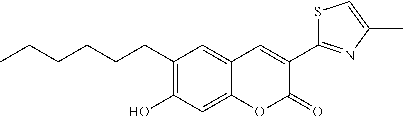 [SMILES: CCCCCCc1cc2cc(-c3nc(C)cs3)c(=O)oc2cc1O]

6-hexyl-7-hydroxy-3-(4-methylthiazol-2-yl)-2H-chromen-2-one, referred to herein as “RCN-010”, or
   a pharmaceutically or cosmetically acceptable salt, solvate, or hydrate thereof.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, a-ketoglutarate, and a-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (e.g., sodium, potassium or lithium) or alkaline earth metal (e.g., calcium) salts of carboxylic acids can also be made.

The compounds of the invention can contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding compounds' enantiomers and stereoisomers, that is, both the stereomerically pure form (e.g., geometrically pure, enantiomerically pure, or diastereomerically pure) and enantiomeric and stereoisomeric mixtures.

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

The compounds of the present invention were obtained from an initial cluster of 10,000 diverse drug like compounds selected for in-house high throughput screening. The initial drug like compounds were selected from an in-house small-molecule database consisting of approximately 10 million compounds based on an innovative machine learning technique drug-like model using FDA approved small molecules as well as those in clinical trial as “drugs” and carcinogens and toxics as “nondrugs”. A validated model was used to screen ˜10 million compounds for drug-like molecules, yielding 10,000 diverse compounds across a variety of structural characteristics.

DM1 inhibitors were identified using an in-house primary DM1 High Throughput Screen coupled to a Secondary Hit validation assay. These assays are the first to identify small molecules that rescue pathology in both patient cells and in DM1 mouse models. The assays have the ability to identify hits that use a variety of different mechanisms to modulate the biology of CUGexp RNAs. No false positives have been obtained thus far.

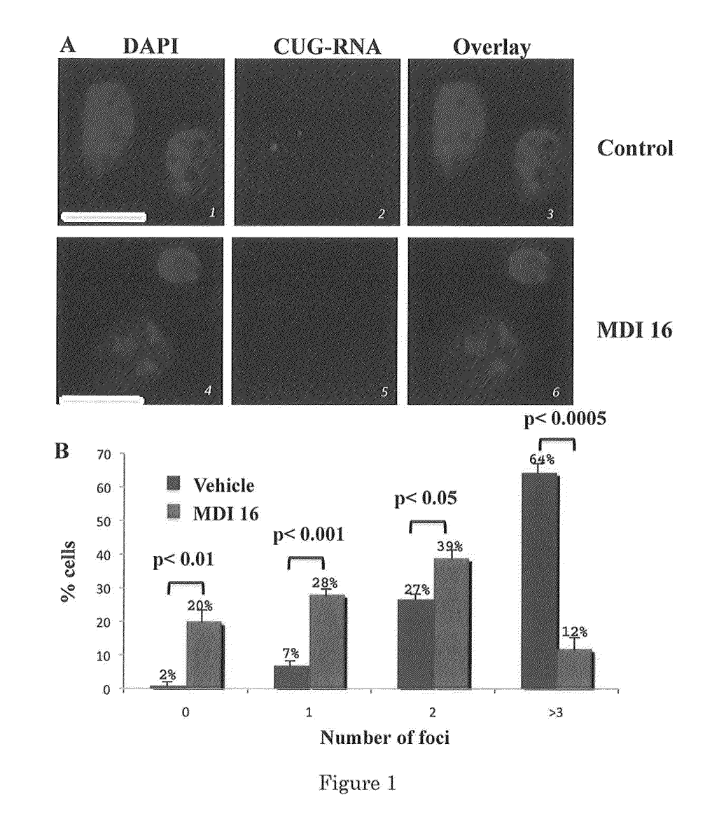

An initial screen of 2500 compounds out of the 10,000 diverse drug like compounds from the small-molecule chemical library resulted in the identification of DM1 inhibitor RCN-001. DM1 myoblasts were then treated with 10 μM of either RCN-001 or the vehicle (DMSO) for 4 days and then examined by FISH using a Cy3 (CAG)₁₀ probe. This analysis revealed that treatment of cells with either 2 or 10 μM RCN-001 results in a dramatic change in the number of CUG-RNA aggregates or foci per cell. Specifically, treatment with 10 μM RCN-001 for 4 days resulted in ˜5-fold decrease in cells containing 3 or more foci and ˜10-fold increase in cells containing no foci (FIG. 1). Consistent with these results, treatment with 2 μM RCN-001 for 4 days resulted in a 2-fold decrease in cells containing 3 or more foci and a 5-fold increase in cells containing no foci (not shown). In addition, this treatment significantly reversed SHARP mislocalization and re-established nuclear SHARP levels in DM1 myoblast (not shown).

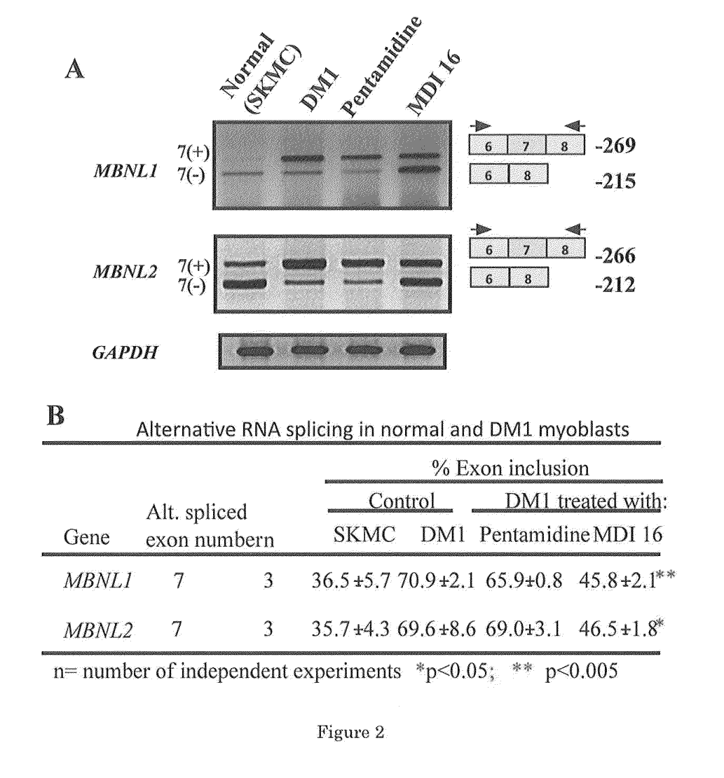

Treatment of DM1 myoblasts with RCN-001 is also sufficient to rescue abnormal RNA splicing. Two RNAs were selected that show abnormal splicing in DM1 myoblasts: MBNL1 and MBNL2. DM1 myoblast were treated with 50 μM RCN-001 for 17 hours after which RNA was isolated and splice variants were analyzed as previously described (20-22). This analysis demonstrates that RCN-001 significantly rescues abnormal splicing of MBNL1 and MBNL2 (21) (FIG. 2). As a control, we treated DM1 cells for 17 hours with 50 μM pentamidine, a molecule that others have recently shown to partially rescue DM1 splicing defects (16), and observed that this molecule did not perform as well RCN-001 with respect to the rescue of these splice defects (FIG. 2).

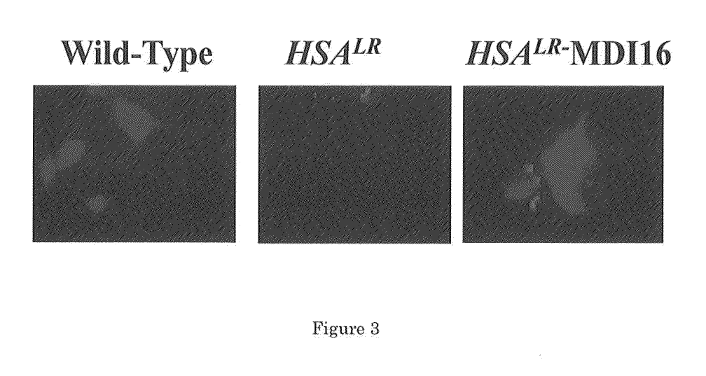

Furthermore, CUG foci, abnormal RNA splicing, myotonia and reduced Clcn1 protein levels are reversed in HSA^{LR} mice treated with RCN-001: Age and gender matched HSA^{LR} mice were treated with either vehicle or 40/mgs/kg of RCN-001 injected i.p. each day for 7 days. After treatment CUG foci were examined and RNA isolated from skeletal muscle was subjected to RT-PCR analysis to examine the efficacy of RCN-001 in correcting the splice defects of Clcn1, lbd3 and Serca-1 RNAs, which are abnormally spliced in DM1 (20). Notably, RCN-001 reduced both the load of the CUG foci and achieved rescue of splice defects that was comparable to results obtained with AON by Thornton and colleagues (FIGS. 3 & 4) (17). This treatment also allowed nuclear SHARP levels to be re-established in HSA^{LR} mice (not shown).

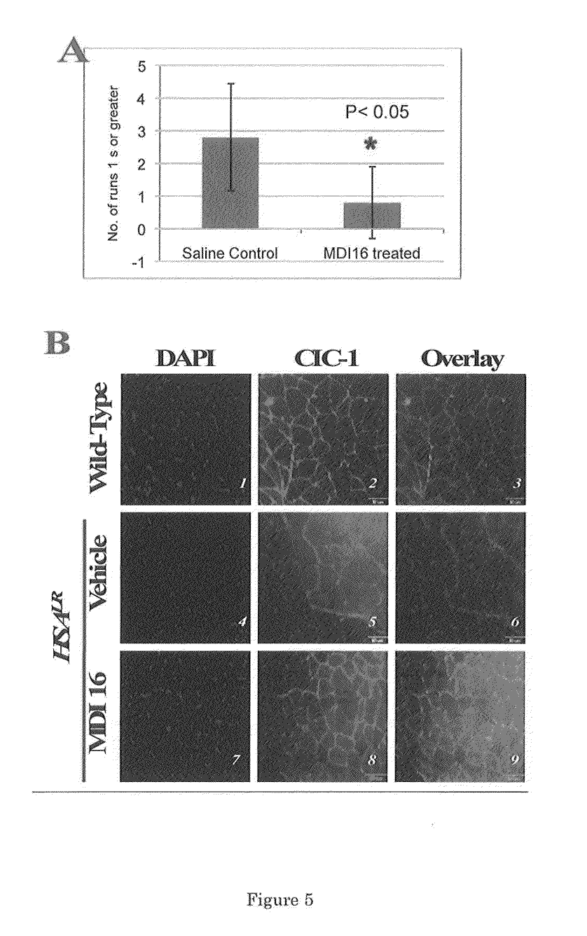

Importantly, RCN-001 reduces the number of myotonic runs of ≧1 second (p<0.05) (FIG. 5), total time of myotonic runs≧1 sec (4.04 s±2.9 vehicle; 0.99 s±1.4 RCN-001; p<0.05) and length of the longest run (1.96 s±0.97 vehicle; 0.73 s±0.71 RCN-001; p<0.05) in the gastrocnemius muscle of HSA^{LR} mice. Similar results were obtained for the paraspinal muscles. Consistent with these results, RCN-001 treatment was found to re-establish normal chloride channel levels in the HSA^{LR} mice (FIG. 5). These results were obtained with a 14 days treatment regiment. Methods are detailed in the legend of FIG. 5.

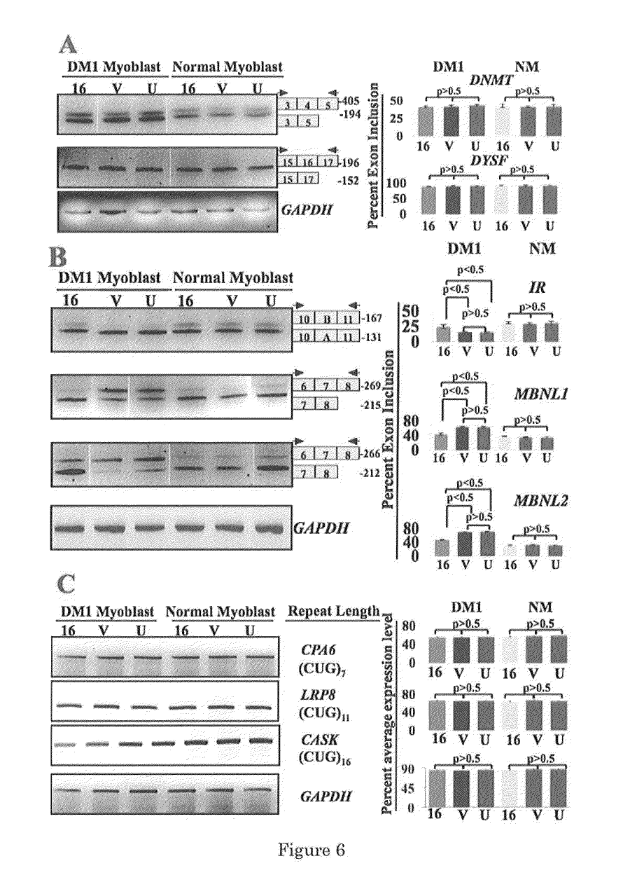

RCN-001 treatment of either normal or DM1 myoblasts and wild-type or HSA^{LR} mice does not alter splice patterns, in sample sets of RNAs that are normally spliced in DM1 (FIG. 6A). RCN-001 treatment does not alter splice patterns of a sample set of RNAs that are abnormally spliced in DM1, in normal myoblasts or in wild-type mice (FIG. 6B). RCN-001 treatment does not show significant off target effects on the steady-state levels of endogenous (CUG)₆₋₂₅ repeat encoding RNAs in wild-type and HSA^{LR} mice or in normal and DM1 myoblasts (FIG. 6C). Results for wild-type and HSA^{LR} mice are not shown.

3D pharmacophore and 2D fingerprinting methods were used to explore RCN-001 analogs. A pharmacophore was generated based on RCN-001 using Catalyst 4.0. The lowest energy conformer of RCN-001 generated by Catalyst was used with the assumption of the bioactive conformer having the lowest energy. The pharmacophore was used to screen an in-house database of 10 million compounds. 2D fingerprinting similarity was carried out in parallel and these two analyses yielded ˜200 compounds.

Representative data is shown in Tables 1-4.

TABLE 1
Activities of substituted chromen-2-one class of compounds.

| | | # of Foci | | | |
|---|---|---|---|---|---|
| | Analogues | (percentage) | | Toxicity | |
| Name | Structure | 0-1 | >2 | Viable | Toxic |
| RCN-002 | 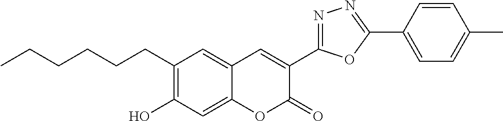 [SMILES: CCCCCCc1cc2cc(-c3nnc(-c4ccc(C)cc4)o3)c(=O)oc2cc1O] | 59 | 41 | +++++ | − |
| RCN-001 | 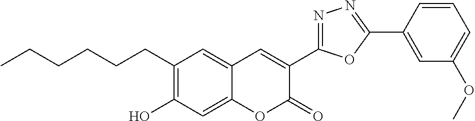 [SMILES: CCCCCCc1cc2cc(-c3nnc(-c4cccc(OC)c4)o3)c(=O)oc2cc1O] | 48 | 52 | ++ | ++++ |
| RCN-002 | 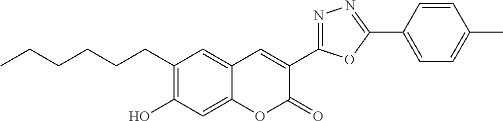 [SMILES: CCCCCCc1cc2cc(-c3nnc(-c4ccc(C)cc4)o3)c(=O)oc2cc1O] | 35 | 65 | +++ | +++ |
| RCN-014 | 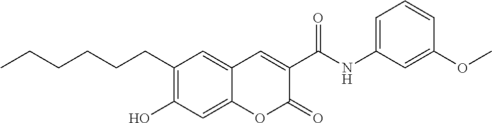 [SMILES: CCCCCCc1cc2cc(C(=O)Nc3cccc(OC)c3)c(=O)oc2cc1O] | 24 | 76 | ++++ | + |
| RCN-003 | 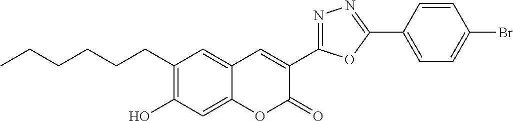 [SMILES: CCCCCCc1cc2cc(-c3nnc(-c4ccc(Br)cc4)o3)c(=O)oc2cc1O] | 31 | 69 | +++ | +++ |
| RCN-004 | 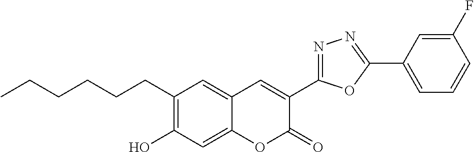 [SMILES: CCCCCCc1cc2cc(-c3nnc(-c4cccc(F)c4)o3)c(=O)oc2cc1O] | 29 | 71 | ++ | +++ |
| RCN-003 | 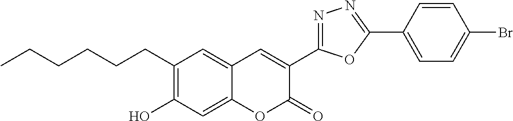 [SMILES: CCCCCCc1cc2cc(-c3nnc(-c4ccc(Br)cc4)o3)c(=O)oc2cc1O] | 21 | 79 | +++ | ++++ |
| RCN-005 | 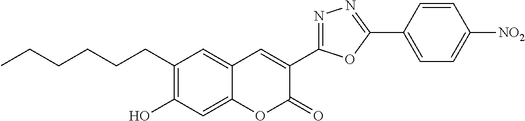 [SMILES: CCCCCCc1cc2cc(-c3nnc(-c4ccc([N+](=O)[O-])cc4)o3)c(=O)oc2cc1O] | 26 | 74 | + | +++ |
| RCN-007 | 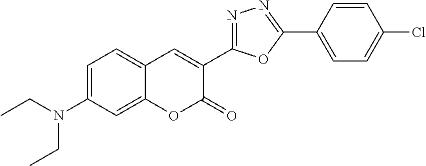 [SMILES: CCN(CC)c1ccc2cc(-c3nnc(-c4ccc(Cl)cc4)o3)c(=O)oc2c1] | 17 | 83 | +++ | +++ |
| RCN-006 | 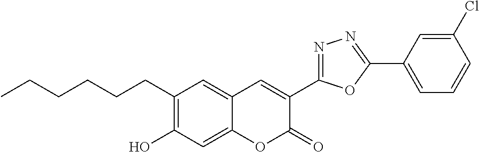 [SMILES: CCCCCCc1cc2cc(-c3nnc(-c4cccc(Cl)c4)o3)c(=O)oc2cc1O] | 16 | 84 | ++ | ++++ |
| RCN--005 | 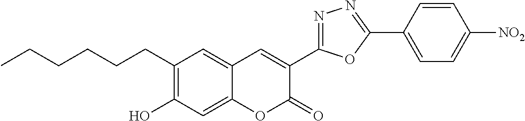 [SMILES: CCCCCCc1cc2cc(-c3nnc(-c4ccc([N+](=O)[O-])cc4)o3)c(=O)oc2cc1O] | 14 | 86 | + | ++++ |
| RCN-006 | 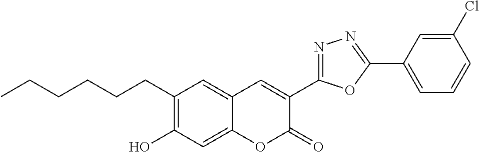 [SMILES: CCCCCCc1cc2cc(-c3nnc(-c4cccc(Cl)c4)o3)c(=O)oc2cc1O] | 9 | 91 | + | +++++ |
| Controls: | | | | | |
| Untreated | DM1 myoblasts | 7 | 93 | +++++ | − |
| Untreated | SKMC normal myoblasts | 100 | 0 | +++++ | − |

DM1 myoblasts were treated with 10 μM of drugs for 4 days and FISH was performed to analyze the change in nuclear foci formation.
N/A means no surviving cells were available for counting.
Foci 0-1: Percentage of cells with no or 1 nuclear foci.
Foci >2: Percentage of cells with 2 or greater nuclear foci.
Toxicity:
Viable: The viability of DM1 myoblasts after 4 days of 10 μM treatment with each drug was estimated by counting the number of surviving cells after
treatment with the vehicle control and the drug.
Viability scale: −: less than 10% cell survival; +: 10-20% cell survival; ++: 30-40% cell survival; +++: 50-60% cell survival; ++++: 70-80% cell survival;
+++++: 90-100% cell survival.
Toxicity: Comparisons were made by assessing the shape and size of the nuclei.
Toxicity scale: −: Nontoxic; +: 10% alterations in nuclei shape and size; ++: 20-30% alterations in nuclei shape and size; +++: 40-50% alterations in nuclei
shape and size; ++++: 60% alterations in nuclei shape and size; +++++: very sick, almost no surviving cells. Quantitative measurement of the toxicity of
the drugs is pending.

TABLE 2
Activities of substituted chromen-2-one class of compounds.

| | | # of Foci | | | |
|---|---|---|---|---|---|
| | Analogues | (percentage) | | Toxicity | |
| Name | Structure | 0-1 | >2 | Viable | Toxic |
| RCN-014 | 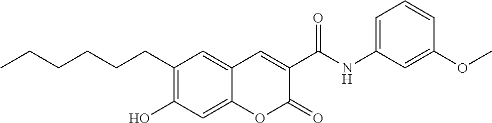 [SMILES: CCCCCCc1cc2cc(C(=O)Nc3cccc(OC)c3)c(=O)oc2cc1O] | 24 | 76 | ++++ | + |
| RCN-015 | 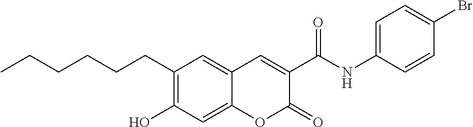 [SMILES: CCCCCCc1cc2cc(C(=O)Nc3ccc(Br)cc3)c(=O)oc2cc1O] | 29 | 71 | +++ | +++ |
| RCN-019 | 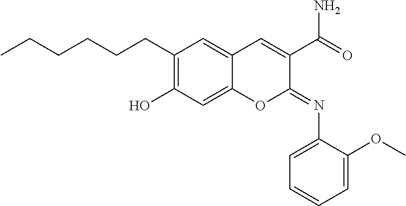 [SMILES: CCCCCCc1cc2cc(C(N)=O)/c(=N/c3ccccc3OC)oc2cc1O] | 14 | 86 | +++ | ++ |
| RCN-016 | 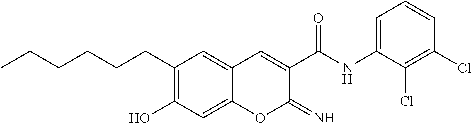 [SMILES: CCCCCCc1cc2cc(C(=O)Nc3cccc(Cl)c3Cl)c(=N)oc2cc1O] | 12 | 88 | +++ | ++ |
| RCN-017 | 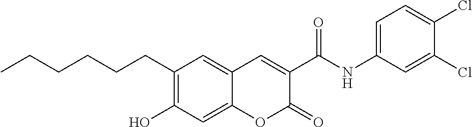 [SMILES: CCCCCCc1cc2cc(C(=O)Nc3ccc(Cl)c(Cl)c3)c(=O)oc2cc1O] | N/A | N/A | − | +++++ |
| RCN-018 | 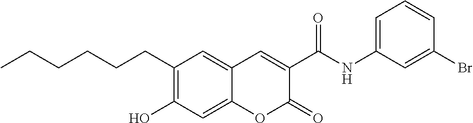 [SMILES: CCCCCCc1cc2cc(C(=O)Nc3cccc(Br)c3)c(=O)oc2cc1O] | N/A | N/A | − | +++++ |
| Controls: | | | | | |
| Untreated | DM1 myoblasts | 7 | 93 | +++++ | − |
| Untreated | SKMC normal myoblasts | 100 | 0 | +++++ | − |

DM1 myoblasts were treated with 10 μM of drugs for 4 days and FISH was performed to analyze the change in nuclear foci formation.
N/A means no surviving cells were available for counting.
Foci 0-1: Percentage of cells with no or 1 nuclear foci.
Foci >2: Percentage of cells with 2 or greater nuclear foci.
Toxicity:
Viable: The viability of DM1 myoblasts after 4 days of 10 μM treatment with each drug was estimated by counting the number of surviving cells after
treatment with the vehicle control and the drug.
Viability scale: −: less than 10% cell survival; +: 10-20% cell survival; ++: 30-40% cell survival; +++: 50-60% cell survival; ++++: 70-80% cell survival;
+++++: 90-100% cell survival.
Toxicity: Comparisons were made by assessing the shape and size of the nuclei.
Toxicity scale: −: Nontoxic; +: 10% alterations in nuclei shape and size; ++: 20-30% alterations in nuclei shape and size; +++: 40-50% alterations in
nuclei shape and size; ++++: 60% alterations in nuclei shape and size; +++++: very sick, almost no surviving
cells. Quantitative measurement of the toxicity of the drugs is pending.

TABLE 3
Activities of substituted chromen-2-one class of compounds.

| | | # of Foci | | | |
|---|---|---|---|---|---|
| | Analogues | (percentage) | | Toxicity | |
| Name | Structure | 0-1 | >2 | Viable | Toxic |
| RCN-008 | 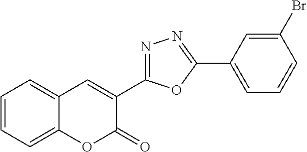 [SMILES: O=c1oc2ccccc2cc1-c1nnc(-c2cccc(Br)c2)o1] | 27 | 23 | +++ | +++ |
| RCN-020 | 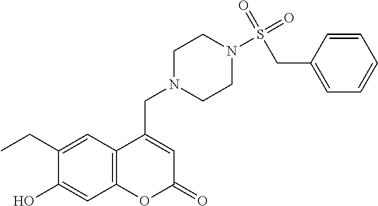 [SMILES: CCc1cc2c(CN3CCN(S(=O)(=O)Cc4ccccc4)CC3)cc(=O)oc2cc1O] | 15 | 85 | +++++ | − |
| RCN-009 | 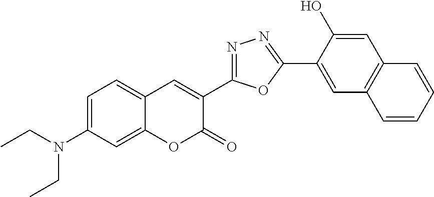 [SMILES: CCN(CC)c1ccc2cc(-c3nnc(-c4cc5ccccc5cc4O)o3)c(=O)oc2c1] | 11 | 89 | ++ | +++ |
| Controls: | | | | | |
| Untreated | DM1 myoblasts | 7 | 93 | +++++ | − |
| Untreated | SKMC normal myoblasts | 100 | 0 | +++++ | − |

DM1 myoblasts were treated with 10 μM of drugs for 4 days and FISH was performed to analyze the change in nuclear foci formation.
N/A means no surviving cells were available for counting.
Foci 0-1: Percentage of cells with no or 1 nuclear foci.
Foci >2: Percentage of cells with 2 or greater nuclear foci.
Toxicity:
Viable: The viability of DM1 myoblasts after 4 days of 10 μM treatment with each drug was estimated by counting the number of surviving
cells after treatment with the vehicle control and the drug.
Viability scale: −: less than 10% cell survival; +: 10-20% cell survival; ++: 30-40% cell survival; +++: 50-60% cell survival;
++++: 70-80% cell survival; +++++: 90-100% cell survival.
Toxicity: Comparisons were made by assessing the shape and size of the nuclei.
Toxicity scale: −: Nontoxic; +: 10% alterations in nuclei shape and size; ++: 20-30% alterations in nuclei shape and size; +++:
40-50% alterations in nuclei shape and size; ++++: 60% alterations in nuclei shape and size; +++++: very sick, almost no surviving cells.
Quantitative measurement of the toxicity of the drugs is pending.

TABLE 4
Activities of substituted chromen-2-one class of compounds.

| | | # of Foci | | | |
|---|---|---|---|---|---|
| | Analogues | (percentage) | | Toxicity | |
| Name | Library/Structure | 0-1 | >2 | Viable | Toxic |
| RCN-010 | 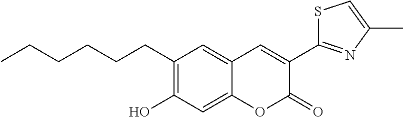 [SMILES: CCCCCCc1cc2cc(-c3nc(C)cs3)c(=O)oc2cc1O] | 17 | 83 | +++++ | − |
| RCN-011 | 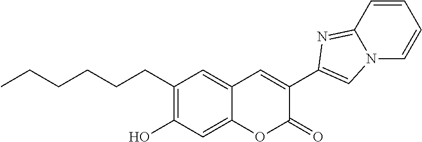 [SMILES: CCCCCCc1cc2cc(-c3cn4ccccc4n3)c(=O)oc2cc1O] | 17 | 83 | +++++ | − |
| RCN-020 | 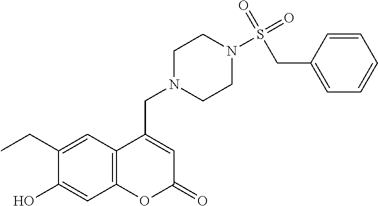 [SMILES: CCc1cc2c(CN3CCN(S(=O)(=O)Cc4ccccc4)CC3)cc(=O)oc2cc1O] | 15 | 85 | +++++ | − |
| RCN-012 | 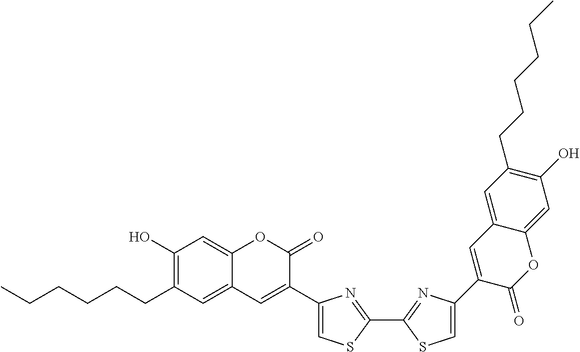 [SMILES: CCCCCCc1cc2cc(-c3csc(-c4nc(-c5cc6cc(CCCCCC)c(O)cc6oc5=O)cs4)n3)c(=O)oc2cc1O] | 14 | 86 | +++++ | − |
| RCN-013 | 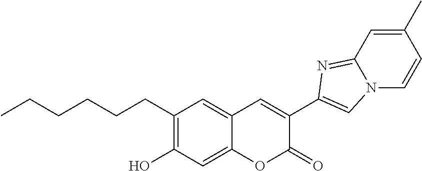 [SMILES: CCCCCCc1cc2cc(-c3cn4ccc(C)cc4n3)c(=O)oc2cc1O] | 13 | 87 | +++ | +++ |
| RCN-010 | 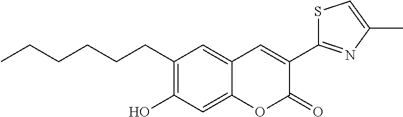 [SMILES: CCCCCCc1cc2cc(-c3nc(C)cs3)c(=O)oc2cc1O] | 10 | 90 | ++ | +++ |
| Controls: | | | | | |
| UNTRD | DM1 myoblasts | 7 | 93 | +++++ | − |
| UNTRD | SKMC normal myoblasts | 100 | 0 | +++++ | − |

DM1 myoblasts were treated with 10 μM of drugs for 4 days and FISH was performed to analyze the change in nuclear foci formation.
N/A means no surviving cells were available for counting.
Foci 0-1: Percentage of cells with no or 1 nuclear foci.
Foci >2: Percentage of cells with 2 or greater nuclear foci.
Toxicity:
Viable: The viability of DM1 myoblasts after 4 days of 10 μM treatment with each drug was estimated by counting the number of surviving cells after treatment
with the vehicle control and the drug.
Viability scale: −: less than 10% cell survival; +: 10-20% cell survival; ++: 30-40% cell survival; +++: 50-60% cell survival; ++++: 70-80% cell survival;
+++++: 90-100% cell survival.
Toxicity: Comparisons were made by assessing the shape and size of the nuclei,
Toxicity scale: −: Nontoxic; +: 10% alterations in nuclei shape and size; ++: 20-30% alterations in nuclei shape and size; +++: 40-50% alterations in nuclei
shape and size; ++++: 60% alterations in nuclei shape and size; +++++: very sick, almost no surviving cells. Quantitative measurement of the toxicity of
the drugs is pending.

RCN-001 can be synthesized according to the method of scheme 1. Other compounds described herein can be synthesized by analogous methods and modified as necessary as would be understood by those of ordinary skill.

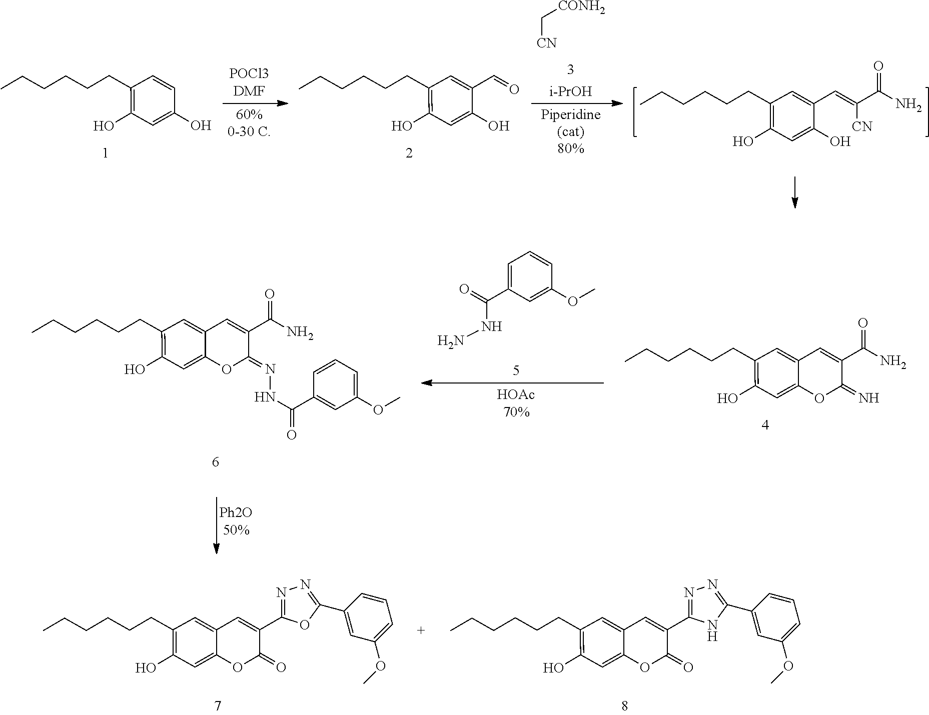

### 5-hexyl-2,4-dihydroxybenzaldehyde (2)

POCl₃ (72 mL) was added to the freshly distilled DMF (90 mL) at −5° C. The reaction mixture was stirred for 1 hr followed by addition of hexylresorcinol (104 g). The reaction mixture was stirred at room temperature for 12 hr. A 30% solution of sodium acetate (500 mL) and ice (1000 mL) were added. The solid product was filtered off and re-crystallized from ethanol to give compound 2 in 73% yield (87.5 g). m.p. 112° C.

### 6-hexyl-7-hydroxy-2-imino-2H-chromene-3-carboxamide (4)

A solution of compound 2 (21 g), cyanoacetamide (8.4 g) and piperidine (1 mL) in isopropanol (300 mL) was heated at reflux for 30 min. After cooling to room temperature, the formed solid was filtered off to provide pure compound 4 in 51% yield (14 g).

### (Z)-6-hexyl-7-hydroxy-2-(2-(3-methoxybenzoyl)hydrazono)-2H-chromene-3-carboxamide (6)

A suspension of hydrazide 5 and 2-iminococumarine 4 (3 g) in acetic acid (10 mL) was heated at 50° C. for 30 minutes. The reaction mixture was cooled to room temperature followed by addition of water. The formed solid was filtered off to provide pure product 6 in 70% yield (4.1 g).

### 6-hexyl-7-hydroxy-3-(5-(3-methoxyphenyl)-1,3,4-oxadiazol-2-yl)-2H-chromen-2-one (7)

A suspension of compound 6 (4 g) in biphenyl ether (30 mL) was heated at 250° C. for 30 minutes. The reaction mixture was cooled to room temperature and the formed solid was filtered off. Crystallization of the crude mixture 7+8 (6-hexyl-7-hydroxy-3-(5-(3-methoxyphenyl)-4H-1,2,4-triazol-3-yl)-2H-chromen-2-one) from acetic acid (50 mL)+DMF (15 mL) gave pure compound 7 (2.3 g) plus compound 8 as side product (10%).

The compounds of the present invention can be formulated as pharmaceutical compositions and administered to a subject in need of treatment, such as a human patient in a variety of forms adapted to the chosen route of administration, i.e., orally or parenterally, by intravenous, intramuscular, topical or subcutaneous routes.

Pharmaceutical compositions of the present invention generally comprise the compounds of the present invention dissolved or dispersed in a suitable carrier. The chosen carrier may be any pharmaceutically acceptable carriers, excipients, or stabilizers which are nontoxic to the cell or mammal being exposed thereto at the dosages and concentrations employed. The physiologically acceptable carrier may be a sterile aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants.

Exemplary pharmaceutical compositions are described below. The pharmaceutical compositions include those suitable for parenteral (including intravenous, subcutaneous, intradermal, intramuscular, and intraarticular), topical (including dermal, transdermal, transmucosal, buccal, sublingual, and intraocular), and rectal administration, although the most suitable route may depend upon, for example, the condition and disorder of the recipient.

The pharmaceutical compositions may be systemically administered, e.g., orally, in combination with a pharmaceutically acceptable vehicle such as an inert diluent or an assimilable edible carrier. They may be enclosed in hard or soft shell gelatin capsules, may be compressed into tablets. For oral therapeutic administration, the active compound may be combined with one or more excipients and used in the form of ingestible tablets, buccal tablets, troches, capsules, elixirs, suspensions, syrups, wafers, and the like. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (e.g., glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze-drying techniques, which yield a powder of the active ingredient plus any additional desired ingredient present in the previously sterile-filtered solutions.

Useful dosages of the compounds can be determined by comparing their in vitro activity, and in vivo activity in animal models. Methods for the extrapolation of effective dosages in mice, and other animals, to humans are known to the art; for example, see U.S. Pat. No. 4,938,949.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Another embodiment of the present invention is directed to methods of treating DM1 and/or preventing the progression of, and/or delaying the onset of DM1 symptoms. The severity of DM1 and symptoms associated with DM1 may be assessed using conventional testing, including conventional clinical methods known in the medical arts. In other embodiments, the method may be a method for reducing the expression of CUGexp RNA, and/or a method of reducing the nuclear CUG-RNA-protein aggregates or foci in DM1 cells. In another embodiment, the method may be a method of reducing the consequence of the expression of toxic CUGexp RNA, i.e. the abnormal splicing, especially of a set of physiologically important RNAs, such as the chloride channel and the IR receptor that have been implicated in the development of myotonia and insulin resistance in DM1 patients. The reduction may be relative to untreated controls or in the subject to be treated before and after treatment with the compounds and/or associated compositions.

Other embodiments of the present invention are directed to methods of treating DM1 and/or preventing the progression of, or delaying the onset of DM1 symptoms. The methods comprise administering to a subject in need thereof a therapeutically effective amounts of the compounds and/or associated compositions of the present invention. A “therapeutically effective amount” of a composition is an amount sufficient to carry out a specifically stated purpose. The effective amount may be determined empirically and in a routine manner in relation to the stated purpose.

The term “subject” as used herein refers to any animal (e.g., a mammal), including, humans, non-human primates, rodents, and the like, which is to be the recipient of a particular treatment. Those in need of treatment include subjects possessing an expansion of the CTG region of DPMK associated with DM1 but who are asymptomatic, those having an expansion of the CTG region of DPMK associated with DM1 but who are asymptomatic but are at risk for developing symptoms of DM1, and those an expansion of the CTG region of DPMK associated with DM1 and are symptomatic.

An assessment of the patient and an associated need for therapy should be performed prior to the start of therapy, and preferably, after the initiation of the treatment methods disclosed herein. Exemplary assessment schedules are as follows: Assessments may be performed at least 14 days from the start of treatment according to the invention, e.g., at least about 7 days or at least about 10 days from the start of treatment. Assessments may be performed at regularly scheduled times post the start of treatment, e.g., every week, every 2 weeks, every 3 weeks, every 4 weeks, every month, every other month, every 3 month, every 6 months, post the start of treatment. Assessments may be performed at least 7 days after each dose where multiple doses are administered, e.g., at least 7 days after each dose, or 14 days after each dose. Assessments may be performed at regularly scheduled times post each dose when multiple doses are administered, e.g., every week, every 2 weeks, every 3 weeks, every 4 weeks, every month, every other month, every 3 months, every 6 months, post each dose. Assessments may be performed at regularly scheduled times post each dose when multiple doses are administered, e.g., at the time of the next dose. The timing of assessment may be altered throughout the course of chronic administration. The timing of assessment may be altered in response to a change in presence or absence of DM1 symptoms or a change in the severity of DM1 symptoms.

In practicing the methods of treatment described herein, the compounds of the present invention may be periodically re-administered. Periodic re-administration can include the administration of more than one dose of an agent over a period of time. Periodic re-administration can include regular administration for an extended period of time. Periodic re-administration can include the administration of therapy over a prolonged period of time, in some cases, for the duration of a subject's lifetime, so that the concentration of the therapeutic agent is maintained at a therapeutically or prophylactically effective level throughout the course of treatment.

The period of time over which periodic re-administration occurs can for the period over which a subject is diagnosed has having an expansion of the CTG region of DPMK associated with DM1 over the lifetime of the subject. The period of time over which periodic re-administration occur can include, but is not limited to, at least 3 months, at least 6 months, at least 1 year, at least 2 years, at least 3 years, at least 4 years, at least 5 years, at least 10 years, at least 15 years, at least 20 years, at least 25 years, at least 30 years, at least 35 years, at least 40 years, at least 45 years, at least 50 years, at least 100 years

Periodic re-administration can include a series of doses which together provide an effective amount for treating DM1 and/or preventing the progression of, or delaying the symptoms associated with DM1. A pharmaceutical composition comprising compounds of the present invention may be administered on dosing schedules tailored to effectuate the treatment of DM1 or the onset of symptoms. The dosing schedule can be dependent on several factors including, the severity of DM1 symptoms the age, body weight, general health, sex and diet of the patient; the time of administration, route of administration, and rate of excretion of the specific composition employed; the duration of the treatment; drugs used in combination or coincidental with the compound and pharmaceutical composition employed; and like factors well known in the medical arts.

Exemplary dosing schedules of the compounds and pharmaceutical compositions may include, once daily, or once weekly, or once every other week, or once monthly, or once every other month, or once every three months, or once every 6 months, or once every 12 months, or once every 18 months, or once every 24 months. The total daily, weekly, or monthly usage of compounds and associated pharmaceutical compositions can be decided by an attending physician within the scope of sound medical judgment.

Even upon improvement of a patient's condition, periodic re-administration of the compounds of the present invention and associated pharmaceutical compositions may be continued. The dosage or frequency of administration, or both, may not be reduced, as a function of DM1 symptoms. The dosage or frequency of administration, or both, may be reduced, as a function of DM1 symptoms, to a level at which the improved condition is retained. Subjects may require intermittent changes in dosage or frequency of administration of treatment on a long-term basis upon any recurrence of DM1 symptoms.

### Materials and Methods

Cell Culture.

Normal myoblasts were purchased from Lanza, Inc. DM1 myoblasts were a gift from Dr. Charles Thornton. Normal and DM I myoblasts were maintained in SkGM Medium (Lonza, Inc.) containing 10% Fetal Bovine Serum and 1% Penicillin-Streptomycin, 5% CO₂ at 37° C. Human Embryonic Kidney 293 Cells (293HEK) were maintained in Dulbecco's Modification of Eagle's Medium (DMEM) from Cellgro supplemented with 10% Fetal Bovine Serum and 1% Penicillin-Streptomycin, 5% CO₂ at 37° C.

Fluorescence In-Situ Hybridization (FISH) and Immunohistochemistry.

CUG-RNA foci were detected by performing FISH on untreated DM1 myoblasts and DM1 myoblasts following treatment. For FISH analysis, DM1 myoblasts were plated on a 24-well clear bottom polystyrene plate with circular glass coverslips followed by treatment. Prior to FISH analysis, cells were fixed using 4% paraformaldehyde in 1×PBS for 20 min at room temperature. FISH analysis was performed using a Cy3 conjugated (CAG)₁₀ oligonucleotide probe (Integrated DNA Technologies, Inc.) as described by Taneja et al, 1995 and Dansithong et al, 2005. CUG-RNA foci were observed as a red signal using fluorescence microscopy. Immunofluorescence was performed as described by Dansithong et al, 2008. Endogenous SHARP was detected using SHARP polyclonal antibodies from Bethyl at a dilution of 1:1000. The secondary antibodies conjugated with FITC (Invitrogen) were used at a 1:2000 dilution. Chloride channel 1 (Clcn1 a.k.a, ClC-1) was detected using ClC-1 polyclonal antibodies from Santa Cruz. The secondary antibodies conjugated with Alexa Flour 488 (Invitrogen) were used at a 1:2000 dilution. FISH analysis in mouse muscle section was carried out as described in Mancodi et al, 2000 (9).

Splicing Assays for RNAs Isolated from Human Myoblasts.

DM1 patient myoblasts was treated with 50 μM of RCN-001 or Pentamidine for 17 h as described by Warf et al, 2009. Total RNA was isolated using the RNAeasy mini kit (Qiagen Inc., USA) following manufacture's protocol. cDNA was synthesized using 5 μg of total RNA using First-Strand cDNA synthesis kit (GE Healthcare). PCR amplification was carried out using 150 ng of cDNA for 35 cycles for each target RNA. The primers used for PCR amplification are as follows—

| | |
|---|---|
| MBNL1 | (For: 5′-GCTGCCCAATACCAGGTCAAC-3′), |
| | (Rev: 5′-TGGTGGGAGAAATGCTGTATGC-3′); |
| MBNL2 | (For: 5′-ACAAGTGACAACACCGTAACCG-3′), |
| | (Rev: 5′-TTTGGTAAAGGATGAAGAGCACC 3′). |
| GAPDH | (For: 5′-TGAAGGTCGGAGTCAACGGATTTGG-3′), |
| | (Rev: 5′-GGAGGCCATGTGGGCCATGAG-3′) |

The relative band intensities were measured using densitometry analyses and the percent of exon inclusion was calculated as [exon inclusion/(exon inclusion+exon exclusion)]×100.

Treatment in HSA^{LR} Mice.

The HSA^{LR} transgenic mice expressing (CTG)₂₅₀ in the 3′UTR of the human skeletal actin mRNA as described in Mankodi et al, 2000 were utilized for this study (a generous gift from Dr. Charles Thornton). Treatment in mice was done by intraperitoneal injection using 40 mg/kg once a day for 7 or 14 days of RCN-001 or vehicle alone. Total RNA was isolated from skeletal muscle of treated HSA^{LR} mice using Trizole (Invitrogen, USA) following the manufacture's protocol. cDNA synthesis and PCR was performed as described in Dansithong et al, 2008. PCR amplification was carried out with the following primers—

| | |
|---|---|
| Clc-1 | (For: 5′ GGAATACCT CACACTCAAGGCC-3′) |
| | (Rev: 5′-CACGGAACACAAAGGCACTGAATGT-3′); |
| Serca-1 | (For: 5′-GCTCATGGTCCTCAAGATCTCAC-3′), |
| | (Rev: 5′-GGGTCAGTGCCTCAGCTTTG-3′); |
| Zasp | (For: 5′-GCAAGACCCTGAAGAGGC-3′), |
| | (Rev: 5′-GTGGGCTGT TACGTTCCGTTT-3′); |
| Gapdh | (For: 5′-AGAGACGGCCGCCGCATCTTCTTGTG-3′), |
| | (Rev: 5′-TCTGGGTGGCAGTGATGGCATGG-3′) |

The relative band intensities were quantified by densitometry analyses and the percent of exon inclusion was calculated as [exon inclusion/(exon inclusion+exon exclusion)]×100.

The examples described herein have been provided in order to demonstrate and further illustrate certain embodiments and aspects of the present invention and are not to be construed as limiting the scope thereof. While such examples are typical of those that might be used, other procedures known to those skilled in the art may alternatively be utilized. Indeed, those of ordinary skill in the art can readily envision and produce further embodiments, based on the teachings herein, without undue experimentation.

### REFERENCES

The following references are incorporated herein by reference:
1. Brook J D, McCurrach M E, Harley H G, Buckler A J, Church D, Aburtani H, Hunter K, Stanton V P, Thirion J-P, Hudson T et al, (1992) Molecular basis of myotonic dystrophy: expansion of a trinucleotide (CTG) repeat at the 3′ end of a transcript encoding a protein kinase family member. Cell 68: 799-808; PMID: 1310900.
2. Taneja K L, McCurrach M, Schalling M, Housman D, Singer R H (1995) Foci of trinucleotide repeat transcripts in nuclei of myotonic dystrophy cells and tissues J. Cell Biol. 128: 995-1002 PMID: 7896884.
3. Miller J W, Urbinati C R, Teng-Umnuay P, Stenberg M G, Byrne B J, C. A. Thornton C A, Swanson M S (2000) Recruitment of human muscleblind proteins to (CUG)(n) expansions associated with myotonic dystrophy. EMBO J 19:4439-4448 PMID: 10970838
4. Fardaei M, Larkin K, Brook J D, Hamshere M G (2001) In vivo co-localisation of MBNL protein with DMPK expanded-repeat transcripts. Nucleic Acids Res. 29: 2766-2771 PMID: 11433021.
5. Mankodi A, Takahashi M P, Jiang H, Beck C L, Bowers W J, Moxley R T, Cannon S C, Thornton C A (2002) Expanded CUG repeats trigger aberrant splicing of ClC-1 chloride channel pre-mRNA and hyperexcitability of skeletal muscle in myotonic dystrophy. Mol. Cell. 1: 35-44 PMID: 12150905
6. Savkur R S, Philips A V, Cooper T A (2001) Aberrant regulation of insulin receptor alternative splicing is associated with insulin resistance in myotonic dystrophy. Nat Genet. 29:40-47 PMID: 11528389
7. Mulders S A, van den Brock W J, Wheeler T M, Croes H J, van Kuik-Romeijn P, de Kimpe, S J, Furling D, Platenburg G J, Gourdon G, Thornton C A, Wiering a B, and Wansink D G. 2009. Triplet-repeat oligonucleotide-mediated reversal of RNA toxicity in myotonic dystrophy. Proc Natl Acad Sci USA 106:13915-13920. PMID: 19667189
8. Gareiss, P C, Sobczak, K., MeNaughton, B R, Palde, P B, Thornton, C A and Miller B L (2008) Dynamic combinatorial selection of molecules capable of inhibiting the (CUG) repeat RNA-MBNL1 interaction in vitro: Discovery of lead compounds targeting myotonic dystrophy. JAGS, 130: 16254-16261. PMID: 18998634.
9. Mankodi A, Logigian E, Callahan L, McClain C, White R, Henderson D, Krym M, Thornton C A (2000) Myotonic dystrophy in transgenic mice expressing an expanded CUG repeat. Science, 289:1769-1773. PMID: 10976074
10. Wang G S, Kearney D L, De Biasi M, Taffet G, Cooper T A (2007) Elevation of RNA-binding protein CUGBP1 is an early event in an inducible heart-specific mouse model of myotonic dystrophy. J. Clin. Invest. 117: 2802-2811 PMID: 17823658.
11. Kanadia, R. N., K. A. Johnstone, A. Mankodi, C. Lungu, C. A. Thornton, D. Esson, A. M. Timmers, W. W. Hauswirth and M. S. Swanson. 2003. A muscleblind knockout model for myotonic dystrophy. Science. 302:1978-1980 PMID: 14671308
12, Du, H., M. S. Cline, R. J. Osborne, D. L. Tuttle, T. A. Clark, J. P. Donohue, M. P. Hall, L. Shine L, M. S. Swanson, C. A. Thornton and M. Ares Jr. (2010) Aberrant alternative splicing and extracellular matrix gene expression in mouse models of myotonic dystrophy. Nat Struct Mal Biol. 17:187-191 PMID: 20098426
13. Kuyumcu-Martinez N M, Wang G S, Cooper T A (2007) Increased steady-state levels of CUGBP1 in myotonic dystrophy 1 are due to PKC-mediated hyperphosphorylation. Mol. Cell. 28:68-78. PMID: 17936705.
14. Dansithong W, Jog S P, Paul S, Mohammadzadeh R, Tring S, Kwok Y, Fry R C, Marjoram P, Comai L, Reddy S. (2011) RNA steady-state defects in myotonic dystrophy are linked to nuclear exclusion of SHARP. EMBO Rep. 12:735-742. PMID:21637295,
15. Yadava R S, Frenzel-McCardell C D, Yu Q, Srinivasan V, Tucker A L, Puymirat J, Thornton C A, Prall O W, Harvey R P, Mahadevan M S (2008) RNA toxicity in myotonic muscular dystrophy induces NKX2-5 expression. Nat. Genet. 40: 61-68 PMID: 18084293
16. Warf, M B., Nakamori, M., Matthys, C M., Thornton, C A. Berglund, J A. (2009) Pentamidine reverses the splicing defects associated with myotonic dystrophy Proc. Natl. Acad. Sci USA 106: 18551-18556. PMID: 19822739.
17. Wheeler T M, Sobczak K, Lueck J D, Osborne R J, Lin X, Dirksen R T, Thornton C A. (2009) Reversal of RNA dominance by displacement of protein sequestered on triplet repeat RNA. Science. 325:336-339. PMID: 19608921,
18. Dayam, R, Aiello, F, Deng, J, Wu, Y, Garofalo, A, Chen, X, Neamati, N. (2006) Discovery of small molecule integrin alphavbeta3 antagonists as novel anticancer agents. J Med Chem 49: 4526-34. PMID: 16854058.
19. Zawahir, Z, Dayam, R, Deng, J, Pereira, C, Neamati, N. (2009) Pharmacophore guided discovery of small-molecule human apurinic/apyrimidinic endonuclease 1 inhibitors. J Med Chem 52: 20-32. PMID: 19072053.
20. Lin X, Miller J W, Mankodi A, Kanadia R N, Yuan Y, Moxley R T, Swanson M S, Thornton C A. (2006) Failure of MBNL1-dependent post-natal splicing transitions in myotonic dystrophy. Hum Mol. Genet. 15: 2087-2097 PMID: 16717059.
21. Dansithong W, Paul S, Comai L, Reddy S (2005) MBNL1 is the primary determinant of focus formation and aberrant insulin receptor splicing in DM1. J. Biol. Chem. 280: 5773-5780. PMID: 15546872.
22, Paul S, Dansithong W, Kim D, Rossi J, Webster N J, Comai L, Reddy S (2006) Interaction of muscleblind, CUG-BP1 and hnRNP H proteins in DM1-associated aberrant IR splicing. EMBO J. 25: 4271-4283 PMID: 16946708.
23. François, V, Klein, A F, Beley, C, Joliet, A, Lernercier, C, Garcia, L, Furling D. (2011) Selective silencing of mutated mRNAs in DM1 by using modified hU7-snRNAs. Nat Struct Mol. Biol. 18:85-7. PMID: 21186365

What is claimed is:

1. A compound having the formula: 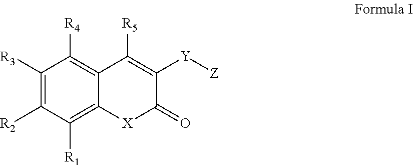
wherein
X is selected from the group consisting of O, N, C, or S,
Y is a homo- or heteroatomic 5-membered ring comprising one or more atoms selected from the group consisting of N, O, S, and C,
Z is an optionally substituted aryl group or optionally substituted heteroaryl, including but not limited to halogenated benzenes, pyridines, substituted benzene, substituted pyridine,
R₂=hydroxy, acyl, alkoxyl, esters, ethers, cyclic ethers, and lactones,
R₃=H, alkyl, an optionally substituted alkyl, aliphatic ether, ester, cyclic unsaturated and aromatic ring groups, and
R₁, R₄ and R₅ are independently selected from the group consisting of hydrogen, halogen, alkyl, and alkoxyl,
or a pharmaceutically or cosmetically acceptable salt, solvate, or hydrate thereof.

2. The compound according to claim 1, wherein the compound has the formula: 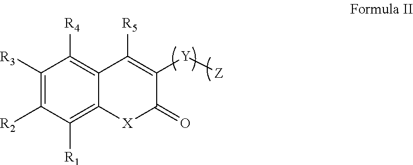
wherein X is O, N, C or S,
R₁ is hydrogen;
R₄ is hydrogen,
Y comprises a five membered heterocyclic ring selected from the group consisting of 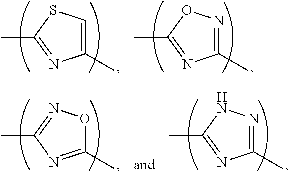
Z is a pyridyl of phenyl group selected from the group consisting of 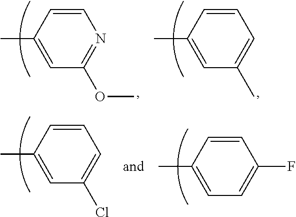
R₂=hydroxy, acyl, alkoxyl, esters, ethers, cyclic ethers, and lactones,
R₃=H, alkyl, an optionally substituted alkyl, aliphatic ether, ester, cyclic unsaturated and aromatic ring groups, and
R₅ are independently selected from the group consisting of hydrogen, halogen, alkyl, and alkoxyl, or
a pharmaceutically or cosmetically acceptable salt, solvate, or hydrate thereof.

3. The compound according to claim 1, wherein the compound is selected from the group consisting of: 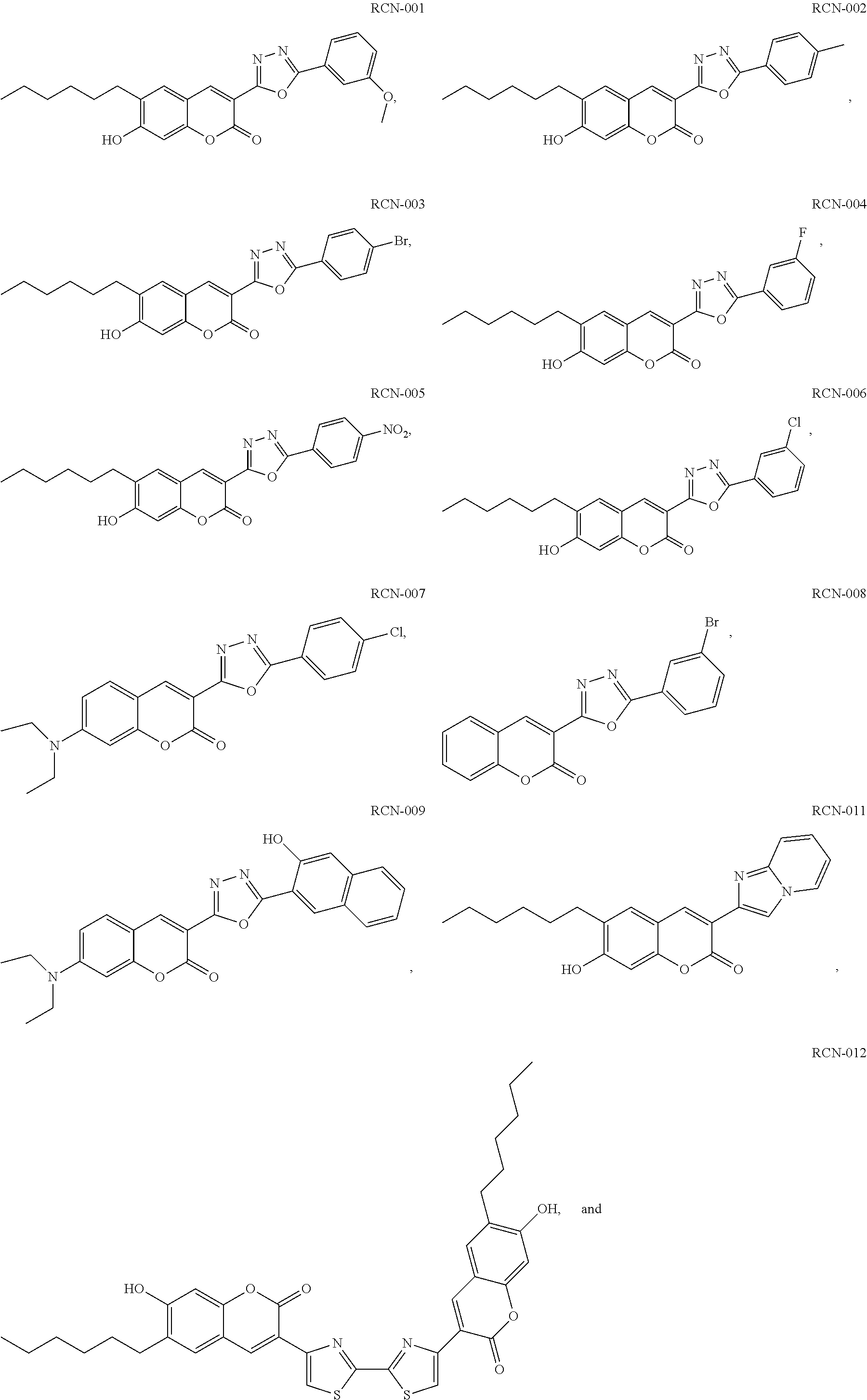 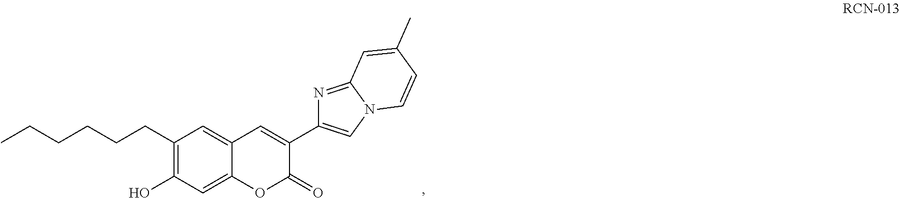 [SMILES: CCCCCCc1cc2cc(-c3cn4ccc(C)cc4n3)c(=O)oc2cc1O]
or a pharmaceutically or cosmetically acceptable salt, solvate, or hydrate thereof.

4. A pharmaceutical composition comprising:
The compound according to claim 1 dispersed or dissolved in a pharmaceutically acceptable carrier.

5. A method of treating myotonic dystrophy type 1 comprising administering to a subject in need thereof an effective amount of the pharmaceutical composition comprising a carrier and the compound according to claim 1.

6. A method of preventing the progression of DM1 symptoms comprising administering to a subject in need thereof a therapeutically effective amounts a pharmaceutical composition comprising a carrier and the compound according to claim 1.

7. A method of preventing the onset of DM1 symptoms comprising administering to a subject in need thereof a therapeutically effective amounts a pharmaceutical composition comprising a carrier and the compound according to claim 1.

8. A pharmaceutical composition comprising:
The compound according to claim 2 dispersed or dissolved in a pharmaceutically acceptable carrier.

9. A method of treating myotonic dystrophy type 1 comprising administering to a subject in need thereof an effective amount of the pharmaceutical composition comprising a carrier and the compound according to claim 2.

10. A method of preventing the progression of DM1 symptoms comprising administering to a subject in need thereof a therapeutically effective amounts a pharmaceutical composition comprising a carrier and the compound according to claim 2.

11. A method of preventing the onset of DM1 symptoms comprising administering to a subject in need thereof a therapeutically effective amounts a pharmaceutical composition comprising a carrier and the compound according to claim 1.

12. A compound having a formula: 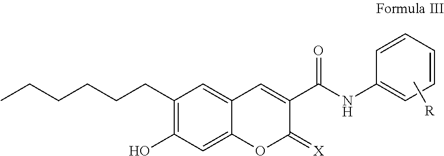
where R indicates a variable substitution of the 6-membered ring and includes mono-, di-, tri-, tetra- and penta-substituted embodiments wherein R is independently selected from the group consisting of halogen, alkyl, alkoxyl, substituted benzene; and
X=O or N, or
a pharmaceutically or cosmetically acceptable salt, solvate, or hydrate thereof.

13. The compound of claim 10, wherein the compound is selected from the group consisting of: 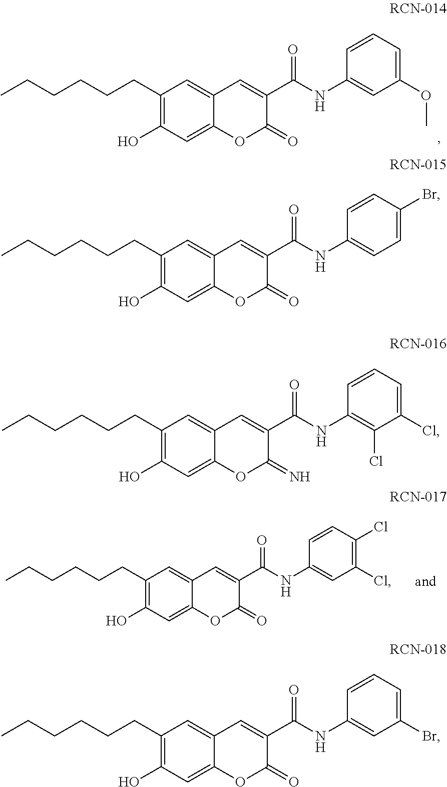 [SMILES: CCCCCCc1cc2cc(C(=O)Nc3cccc(OC)c3)c(=O)oc2cc1O ; CCCCCCc1cc2cc(C(=O)Nc3ccc(Br)cc3)c(=O)oc2cc1O ; CCCCCCc1cc2cc(C(=O)Nc3cccc(Cl)c3Cl)c(=N)oc2cc1O ; CCCCCCc1cc2cc(C(=O)Nc3ccc(Cl)c(Cl)c3)c(=O)oc2cc1O ; "and" ; CCCCCCc1cc2cc(C(=O)Nc3cccc(Br)c3)c(=O)oc2cc1O]
or a pharmaceutically or cosmetically acceptable salt, solvate, or hydrate thereof.

14. A pharmaceutical composition comprising:
The compound according to claim 12 dispersed or dissolved in a pharmaceutically acceptable carrier.

15. A method of treating myotonic dystrophy type 1 comprising administering to a subject in need in need thereof an effective amount of the pharmaceutical composition comprising a carrier and the compound according to claim 12.

16. A method of preventing the progression of DM1 symptoms comprising administering to a subject in need thereof a therapeutically effective amounts a pharmaceutical composition comprising a carrier and the compound according to claim 12.

17. A method of preventing the onset of DM1 symptoms comprising administering to a subject in need thereof a therapeutically effective amounts a pharmaceutical composition comprising a carrier and the compound according to claim 12.

18. A compound selected from the group consisting of: 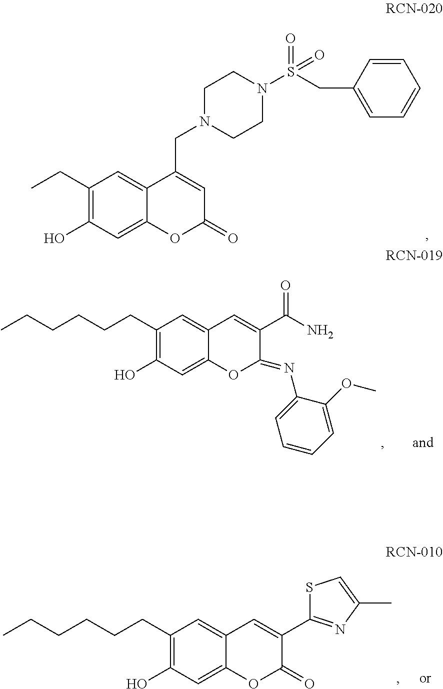
a pharmaceutically or cosmetically acceptable salt, solvate, or hydrate thereof.

19. A pharmaceutical composition comprising:
The compound according to claim 2 dispersed or dissolved in a pharmaceutically acceptable carrier.

20. A method of treating myotonic dystrophy type 1 comprising administering to a subject in need thereof an effective amount of the pharmaceutical composition comprising a carrier and the compound according to claim 2.

21. A method of preventing the progression of DM1 symptoms comprising administering to a subject in need thereof a therapeutically effective amounts a pharmaceutical composition comprising a carrier and the compound according to claim 2.

22. A method of preventing the onset of DM1 symptoms comprising administering to a subject in need thereof a therapeutically effective amounts a pharmaceutical composition comprising a carrier and the compound according to claim 1.


SEQUENCE LISTING (14 sequences)

SEQ ID NO: 1 - DNA, 21 bp, Artificial Sequence
  Synthetic oligonucleotide
gctgcccaataccaggtcaac

SEQ ID NO: 2 - DNA, 22 bp, Artificial Sequence
  Synthetic oligonucleotide
tggtgggagaaatgctgtatgc

SEQ ID NO: 3 - DNA, 22 bp, Artificial Sequence
  Synthetic oligonucleotide
acaagtgacaacaccgtaaccg

SEQ ID NO: 4 - DNA, 23 bp, Artificial Sequence
  Synthetic oligonucleotide
tttggtaaaggatgaagagcacc

SEQ ID NO: 5 - DNA, 25 bp, Artificial Sequence
  Synthetic oligonucleotide
tgaaggtcggagtcaacggatttgg

SEQ ID NO: 6 - DNA, 21 bp, Artificial Sequence
  Synthetic oligonucleotide
ggaggccatgtgggccatgag

SEQ ID NO: 7 - DNA, 22 bp, Artificial Sequence
  Synthetic oligonucleotide
ggaatacctcacactcaaggcc

SEQ ID NO: 8 - DNA, 25 bp, Artificial Sequence
  Synthetic oligonucleotide
cacggaacacaaaggcactgaatgt

SEQ ID NO: 9 - DNA, 23 bp, Artificial Sequence
  Synthetic oligonucleotide
gctcatggtcctcaagatctcac

SEQ ID NO: 10 - DNA, 20 bp, Artificial Sequence
  Synthetic oligonucleotide
gggtcagtgcctcagctttg

SEQ ID NO: 11 - DNA, 18 bp, Artificial Sequence
  Synthetic oligonucleotide
gcaagaccctgaagaggc

SEQ ID NO: 12 - DNA, 21 bp, Artificial Sequence
  Synthetic oligonucleotide
gtgggctgttacgttccgttt

SEQ ID NO: 13 - DNA, 26 bp, Artificial Sequence
  Synthetic oligonucleotide
agagacggccgccgcatcttcttgtg

SEQ ID NO: 14 - DNA, 23 bp, Artificial Sequence
  Synthetic oligonucleotide
tctgggtggcagtgatggcatgg